(19) United States
(12) Patent Application Publication
(10) Pub. No.: US 2019/0343376 A1
(43) Pub. Date: Nov. 14, 2019

(54) METHODS AND APPARATUS FOR A CLEANING SHEATH FOR AN ENDOSCOPE

(71) Applicant: Hao Kenith Fang, Scottsdale, AZ (US)

(72) Inventor: Hao Kenith Fang, Scottsdale, AZ (US)

(21) Appl. No.: 16/401,319

(22) Filed: May 2, 2019

Related U.S. Application Data
(60) Provisional application No. 62/670,205, filed on May 11, 2018.

(51) Int. Cl. A61B 1/12; A61B 1/00

(52) U.S. Cl. CPC A61B 1/126; A61B 1/015; A61B 1/00119; A61B 1/00142

(57) ABSTRACT

A cleaning sheath for an endoscope according to various aspects of the present technology includes an open ended sheath coupled to a fluid line. The sheath is configured to receive an endoscope within the interior of the sheath wherein both the sheath and the endoscope may be inserted into a trocar during use. The sheath is further configured to direct a cleaning fluid onto the optics end of the endoscope to clear away any visual obstructions without requiring the removal of the endoscope from the trocar.

### CROSS-REFERENCES TO RELATED APPLICATIONS

This application claims the benefit of U.S. Provisional Patent Application No. 62/670,205, filed May 11, 2018, and incorporates the disclosure of the application by reference.

### BACKGROUND OF THE TECHNOLOGY

Endoscopes are used in a variety of surgical procedures including endscopy, laparoscopy, and thoracoscopy to investigate symptoms in the digestive system, cardiovascular system, and the sinuses. A common frustration experienced in procedures is the altered and obstructed visualization caused by debris and bodily fluids that come into contact with the distal fiberoptic tip of the endoscope. In addition, upon first insertion of the endoscope into a warm body cavity, the lens of the optics tip may fog similar to eye goggles when swimming or snorkeling. When this occurs, it typically requires the removal of the endoscope from the body cavity to remove the obstructing substance manually. Removing and reinserting the endoscope wastes valuable O.R. time and is potentially dangerous when visualization is compromised during a critical point of an operation/procedure.

Existing solutions directed towards maintaining a clear field of vision after the tip of the endoscope has been inserted into a body cavity include devices that do not function with existing sizes of associated medical devices used during the surgical procedure such as a trocar. These devices often also require separate pumps, motors, electronics, and switches to function properly. Each of these devices increases the number of equipment located where the surgical procedure is being performed taking up space and increasing costs. Further, introducing additional components during the surgical procedure results in increased complexity and requires additional training by medical staff.

### SUMMARY OF THE TECHNOLOGY

A cleaning sheath for an endoscope according to various aspects of the present technology includes an open ended sheath coupled to a fluid line. The sheath is configured to receive an endoscope within the interior of the sheath wherein both the sheath and the endoscope may be inserted into a trocar during use. The sheath is further configured to direct a cleaning fluid onto the optics end of the endoscope to clear away any visual obstructions without requiring the removal of the endoscope from the trocar.

### BRIEF DESCRIPTION OF THE DRAWINGS

A more complete understanding of the present technology may be derived by referring to the detailed description and claims when considered in connection with the following illustrative figures. In the following figures, like reference numbers refer to similar elements and steps throughout the figures.

FIG. 1 representatively illustrates an endoscope sheath in accordance with an exemplary embodiment of the present technology;

FIG. 2 representatively illustrates a cross-sectional view across line 2-2 of FIG. 1 in accordance with an exemplary embodiment of the present technology;

FIG. 3 representatively illustrates the endoscope sheath coupled to a syringe with an endoscope partially inserted into the endoscope sheath in accordance with an exemplary embodiment of the present technology;

FIG. 4 representatively illustrates a top view of a fluid hub of the endoscope sheath in accordance with an exemplary embodiment of the present technology;

FIG. 5 representatively illustrates a cross-sectional view across line 5-5 of FIG. 4 in accordance with an exemplary embodiment of the present technology;

FIG. 6 representatively illustrates an interior side view of a distal end of an alternative embodiment of the endoscope sheath in accordance with an exemplary embodiment of the present technology;

FIG. 7 representatively illustrates a cross-sectional view of the endoscope sheath in accordance with an exemplary embodiment of the present technology;

FIG. 8 representatively illustrates a detailed view of Section 8 of FIG. 7 in accordance with an exemplary embodiment of the present technology;

FIG. 9 representatively illustrates a cross-sectional view across line 9-9 of FIG. 6 in accordance with an exemplary embodiment of the present technology;

FIG. 10 representatively illustrates a cross-sectional view across line 10-10 of FIG. 6 in accordance with an exemplary embodiment of the present technology;

FIG. 11 representatively illustrates a detailed view of Section 11 of FIG. 10 in accordance with an exemplary embodiment of the present technology

FIG. 12 representatively illustrates an optics end of an endoscope being inserted into the endoscope sheath in accordance with an exemplary embodiment of the present technology;

FIG. 13 representatively illustrates the optics end of the endoscope inserted part way through the endoscope sheath in accordance with an exemplary embodiment of the present technology;

FIG. 14 representatively illustrates the optics end of the endoscope being inserted fully into the endoscope sheath in accordance with an exemplary embodiment of the present technology;

FIG. 15 representatively illustrates the optics end of the endoscope and the endoscope sheath being inserted into a trocar in accordance with an exemplary embodiment of the present technology;

FIG. 16 representatively illustrates the optics end of the endoscope and the endoscope sheath being fully inserted into the trocar in accordance with an exemplary embodiment of the present technology;

FIG. 17 representatively illustrates a syringe being connected to a fluid line of the endoscope sheath in accordance with an exemplary embodiment of the present technology;

FIG. 18 representatively illustrates a display of the optics end of the endoscope in accordance with an exemplary embodiment of the present technology;

FIG. 19 representatively illustrates the display of the optics end of the endoscope with a visual obstruction in accordance with an exemplary embodiment of the present technology;

FIG. 20 representatively illustrates the display of the optics end of the endoscope at the beginning of a flush cycle in accordance with an exemplary embodiment of the present technology;

FIG. 21 representatively illustrates the display of the optics end of the endoscope during the flush cycle in accordance with an exemplary embodiment of the present technology; and

FIG. 22 representatively illustrates the display of the optics end of the endoscope at the completion of the flush cycle in accordance with an exemplary embodiment of the present technology.

Elements and steps in the figures are illustrated for simplicity and clarity and have not necessarily been rendered according to any particular sequence. For example, steps that may be performed concurrently or in a different order are illustrated in the figures to help to improve understanding of embodiments of the present technology.

### DETAILED DESCRIPTION OF EXEMPLARY EMBODIMENTS

The present technology may be described in terms of functional block components and various processing steps. Such functional blocks may be realized by any number of components configured to perform the specified functions and achieve the various results. For example, the present technology may employ various materials, coupling mechanisms, dimensions, and geometries, which may carry out a variety of operations suited to a selective attachment to or use with an endoscope, trocar, or syringe. In addition, the technology described is merely one exemplary application for the invention. Further, the present technology may employ any number of conventional techniques for flushing, cleaning, or otherwise clearing debris such as bodily fluids and fog from optical devices.

Methods and apparatus for an endoscope sheath according to various aspects of the present technology may operate in conjunction with any type of endoscope, fiberoptic video capture system, or micro-camera. Various representative implementations of the present technology may be applied to any type of viewing device that is insertable or otherwise intended for use within a body during a medical procedure that may be subjected to various types of visual obstructions.

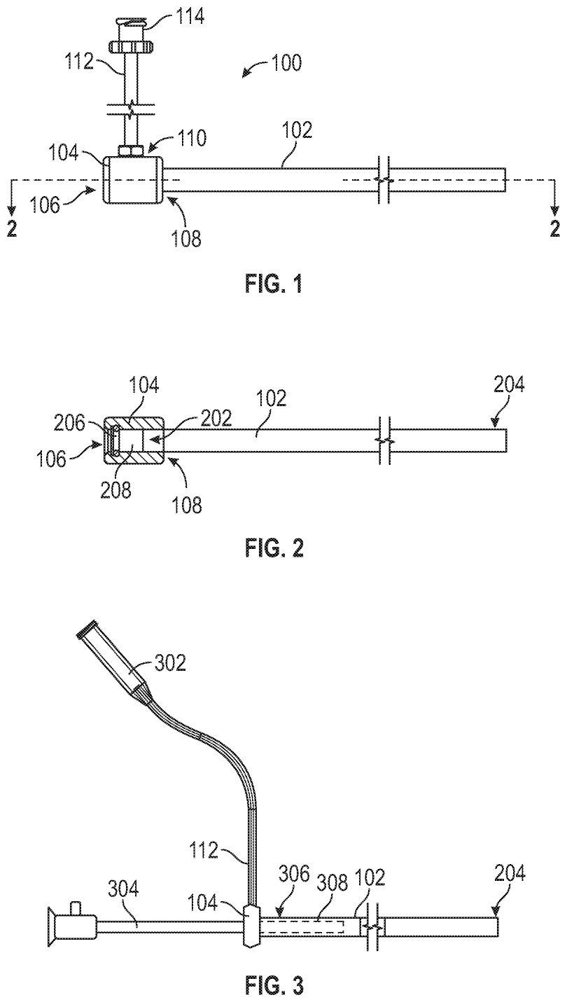

Referring to FIGS. 1-3, a cleaning sheath 100 may comprise a generally tubular shaped body 102 having a distal end 204 and a proximal end 202. At least a portion of an interior of the body 102 and the distal end 204 are configured to receive a viewing device such as an endoscope 304. The body 102 may form a lumen extending between the proximal and distal ends 202, 204 to both receive the endoscope and allow flow of a cleaning fluid, such as a saline solution, from the proximal end 202 to the distal end 204. A fluid hub 104 may be coupled to the proximal end 202 of the body 102 and be in fluid communication with the lumen. A fluid line 112 may be coupled to the fluid hub 104 and be configured to provide a conduit path for the cleaning fluid.

The body 102 may comprise any suitable size or shape and may be selected according to any applicable criteria such as a size of the viewing device the body 102 will be positioned over. For example, the body 102 may comprise an internal diameter of between 2 mm and 14 mm to allow both the endoscope 304 and the body 102 to be positioned within a trocar (not shown) as commonly used in medical procedures.

The internal diameter of the body 102 may be sized slightly larger than the outer diameter of the endoscope 304 to allow the cleaning fluid to flow completely around an outer surface 308 of the endoscope towards the distal end 204 of the body 102. For example, and in one embodiment where the endoscope 304 comprises a 5 mm thorascope having an outer working diameter of about 5.4 mm, the body 102 may comprise an inner diameter of between 5.42 mm and 5.75 mm. The larger diameter of the body 102 relative to the endoscope 304 creates a fluid conduit 306 that extends completely around the outer surface 308 of the endoscope and along the length of the body 102 between the proximal and distal ends 202, 204 in which the cleaning fluid may flow.

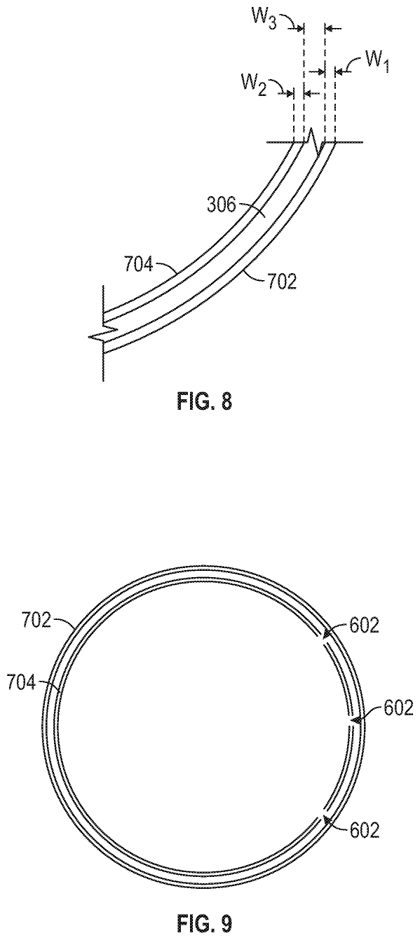

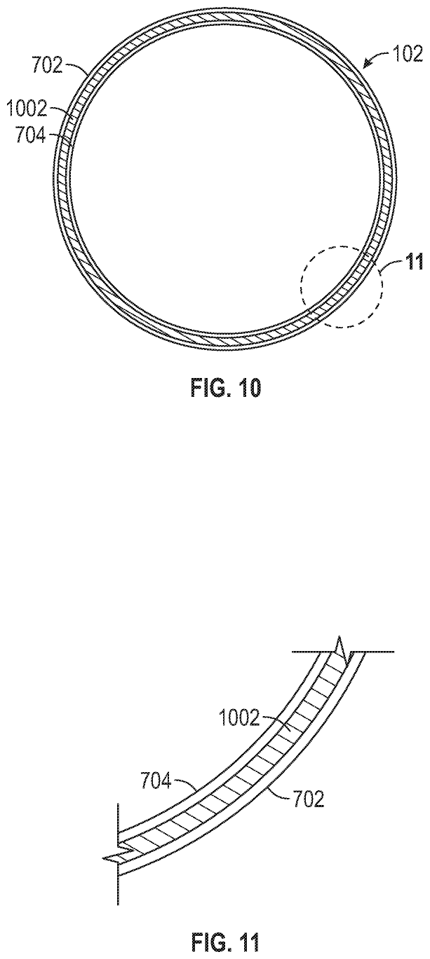

In an alternate embodiment, and referring now to FIGS. 7-11, the body 102 may comprise an outer wall 702 disposed around an inner wall 704 such that the fluid conduit 306 is formed between the inner and outer walls 702, 704 running along the entire length of the body 102. The total wall thickness of the body 102 may comprise the sum of the thickness of: the outer wall 702 (W₁), the inner wall 704 (W₂), and the fluid conduit 306 (W₃).

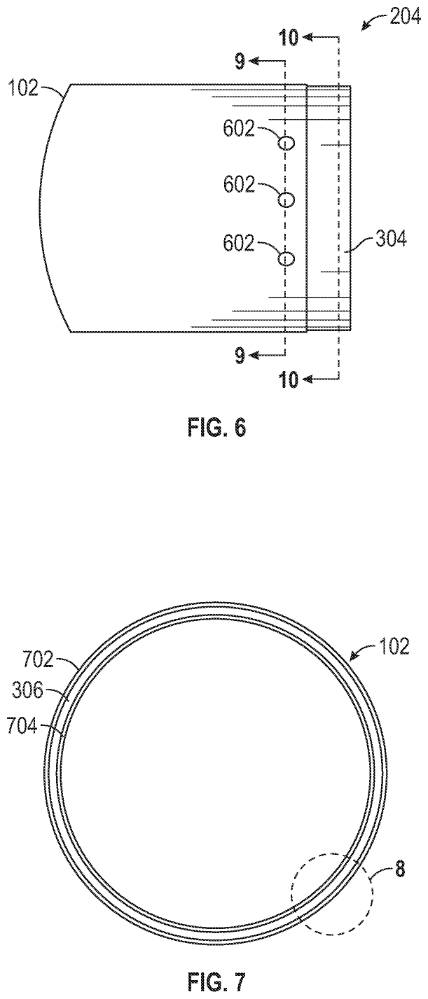

Referring now to FIGS. 6 and 9, the distal end 204 of the body 102 may comprise a plurality of openings 602 positioned around at least a portion of the periphery of the inner wall 704 at predetermined locations to allow the cleaning fluid to be ejected inward from the body 102 towards the viewing device (not shown). The plurality of openings 602 may be positioned at or proximate the end most portion of the distal end 204.

Referring now to FIGS. 7, 10, and 11, the inner wall 704 and the outer wall 702 may be coupled together at the distal end 204. The inner and outer walls 704, 702 may be coupled together in any suitable manner to create a seal 1002 at the distal end 204 of the body 102. The presence of the seal 1002 at the distal end 204 forces the cleaning fluid to flow outwards from the plurality of openings 602.

The seal 1002 may comprise any suitable system or device capable of coupling or otherwise bonding the inner and outer walls 704, 702 together. For example, in one embodiment, the inner and outer walls 704, 702 may be fused or otherwise bonded together with an adhesive that creates the seal 702.

The body 102 may comprise a semi-flexible material suitable material or combinations of materials suitable for use inside the human body such as natural or synthetic polymers, thermoplastics, or metals. For example, in one embodiment, the body 102 may comprise a polyimide polymer adapted to withstand elevated temperatures associated with a light source of the viewing device. In an alternative embodiment, the body 102 may comprise polyether ether ketone (PEEK).

The distal end 204 may be terminated in any suitable manner to allow the viewing device to function properly. For example, the distal end 204 may be terminated at an angle relative to a longitudinal axis of the body 102 to accommodate viewing devices having an angled terminal end of up to 45°. Alternatively, the distal end 204 may be terminated at a substantially right angle to the body 102 to accommodate viewing devices having a visualization angle of 0°.

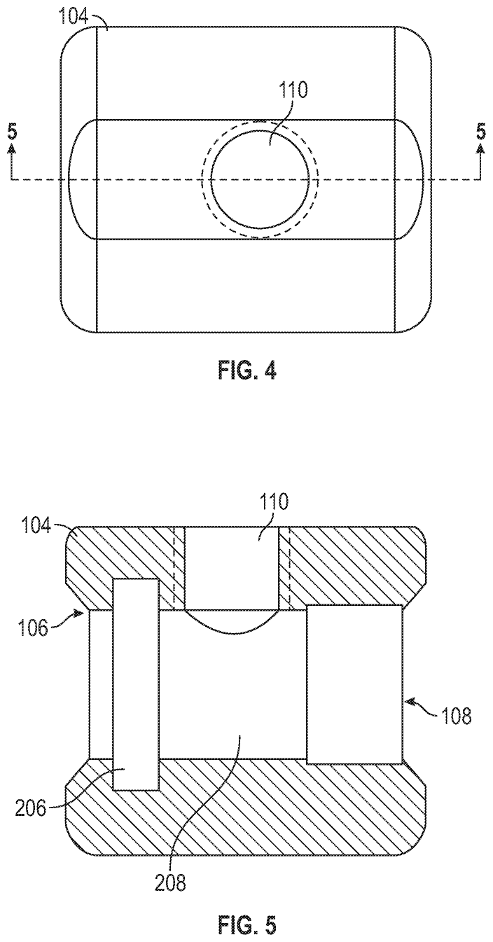

Referring now to FIGS. 1, 2, 4, and 5, the fluid hub 104 provides fluid link between the fluid line 112 and the lumen of the body 102. The fluid hub 104 comprises a hub lumen 208 extending between opposing first and second open ends 106, 108 that form an insertion path for the viewing device into the lumen of the body 102. The second open end 108 of the fluid hub 104 receives and is coupled to the proximal end 202 of the body.

An inlet port 110 is open to the hub lumen 208 and is disposed between the first and second open ends 106, 108 and is coupled to the fluid line 112. The fluid line 112 may be coupled to the inlet port 110 by any suitable method. In one embodiment, the inlet port 110 may be configured with a compression fitting configured to create a seal between the fluid line 112 and the interior of the fluid hub 104. In an alternative embodiment, the inlet port 110 may comprise an opening approximately equal to or slightly less than an outer diameter of a first end of the fluid line 112 with an edge configured to create a seal against the fluid line 112 when an end of the fluid line 112 is inserted through the opening.

The first open end 106 of the fluid hub 104 may be configured to create a seal against the viewing device when it is inserted through the fluid hub 104 to prevent fluid from exiting out of the first open end 106 during use. For example, the first open end 106 may comprise a sealing element 206 positioned between an outer edge of the first open end 106 and an inlet port 110. The sealing element 206 may comprise any suitable system or device for forming a seal against an outer surface of the viewing device and the hub lumen 208. In one embodiment, the sealing element may comprise an o-ring having an inner diameter approximately equal to that of the diameter of the outer surface of the viewing device that is intended to be used with the cleaning sheath 100.

Referring again to FIGS. 1-3, the fluid line 112 may comprise any suitable system or device for creating a fluid conduit path between a cleaning fluid supply source and the fluid hub 104. For example, the fluid line 112 may comprise a flexible tube made from a polymer. The fluid line 112 may comprise any suitable length or diameter. For example, in one embodiment, the fluid line 112 may comprise a length of between about 50 mm and about 300 mm and comprise a diameter between about 2.5 mm and about 25 mm.

As described above, the first end of the fluid line 112 may be coupled to the inlet port 110 at or near the proximal end 202 of the body 102. A second end of the fluid line 112 may be configured to be connected to the cleaning fluid supply. For example, in one embodiment, the second end of the fluid line 112 comprise a leur lock 114 that may be selectively connected to an end of a syringe 302 containing the cleaning fluid.

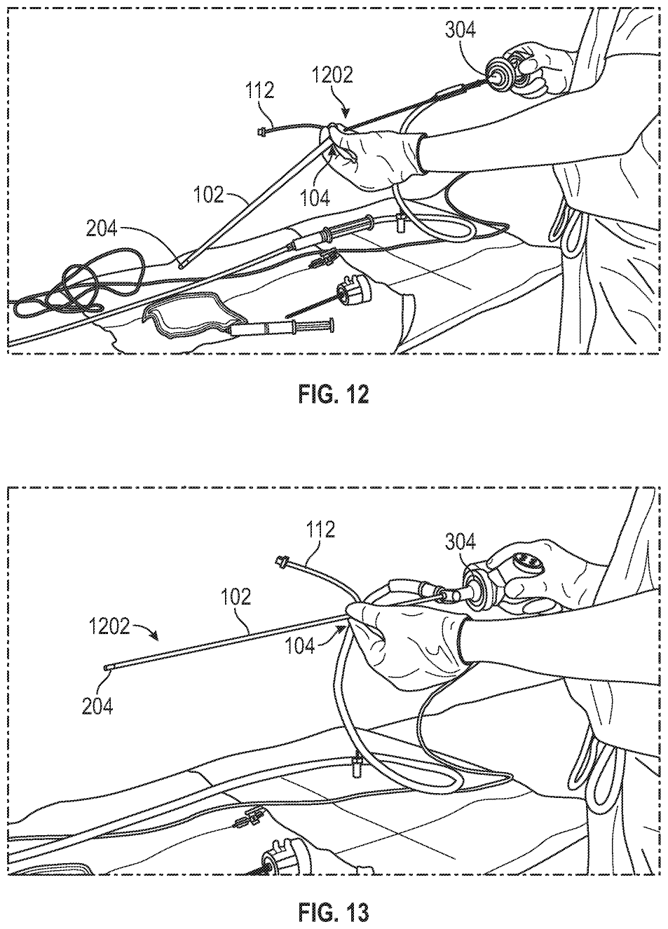

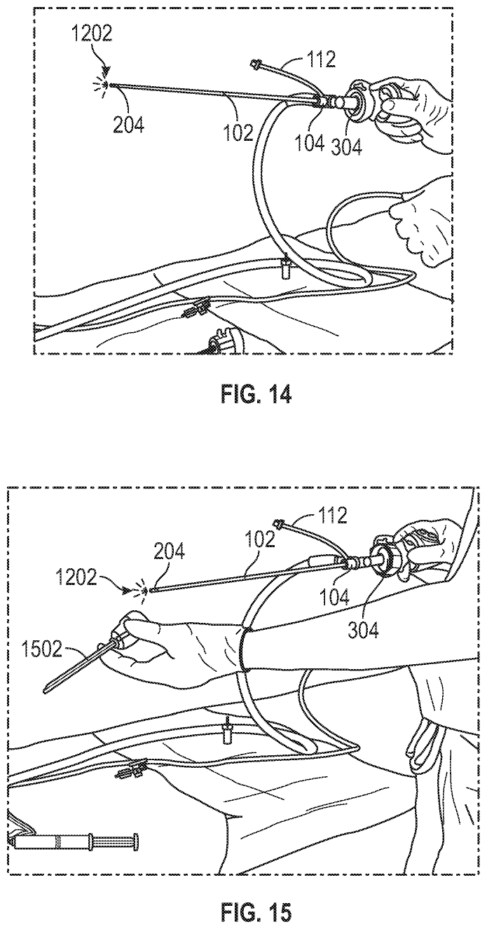

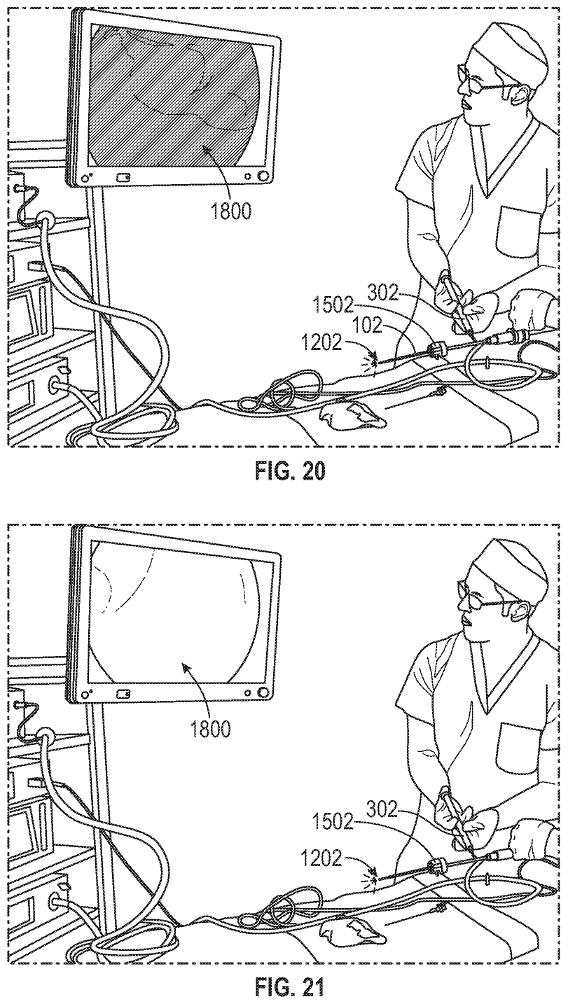

Referring now to FIGS. 12-22, in use the cleaning sheath 100 is used in conjunction with an endoscope 304 during the medical procedure and is configured to direct a cleaning fluid towards the optics end 1202 of the endoscope 304 to clear away any visual obstruction. With particular reference to FIGS. 12-14, the optics end 1202 may be inserted into the first end of the fluid hub 104 and pushed towards the distal end 204 of the body 102 until the optics end 1202 is positioned at the terminal opening of the distal end 204.

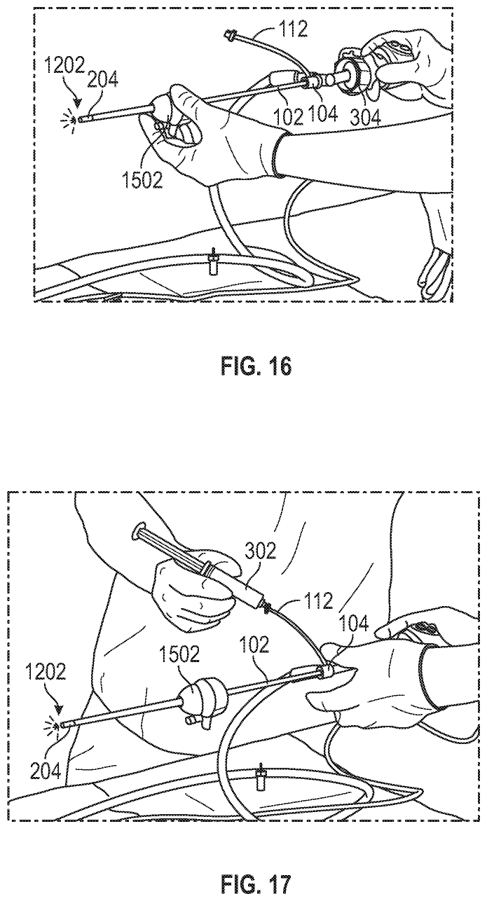

Referring now to FIGS. 15 and 16, during the medical procedure the combined endoscope 304 and the distal end 204 of the cleaning sheath 100 may be inserted through a trocar 1502 and subsequently into the body of the patient. As shown in FIG. 17, a syringe 302 containing the fluid supply may be connected to the second end of the fluid line 112.

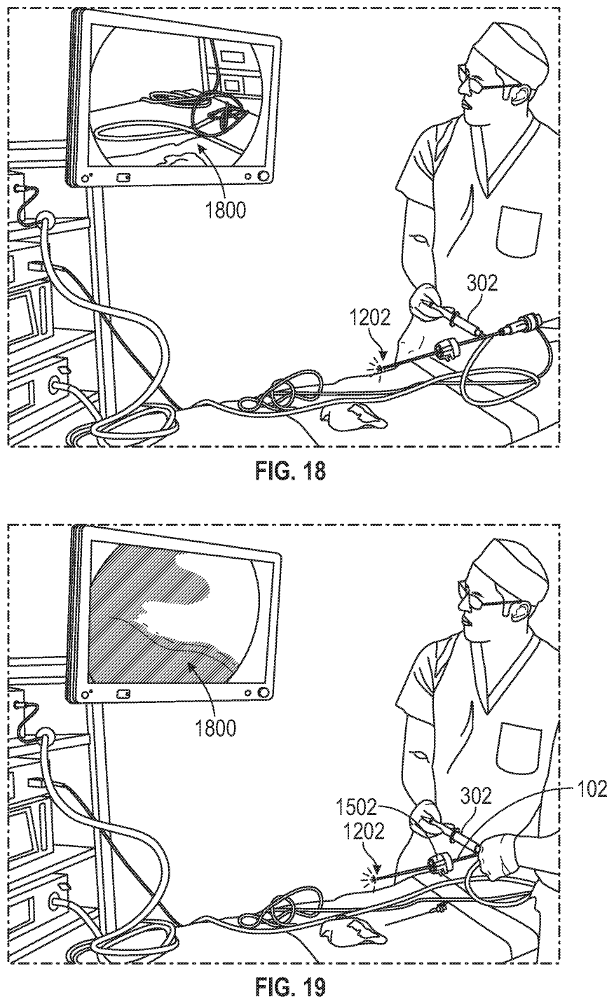

As shown in FIG. 18 the optics end 1202 of the endoscope provides an image to a display device 1800 that may be viewed by the person performing the medical procedure. During the procedure, the optics end 1202 may become obstructed by coming into contact with debris or a bodily fluid or due to a difference in temperature between the optics end 1202 and the internal temperature of the body. This results in an obstructed field of view on the display device 1800 as shown in FIG. 19.

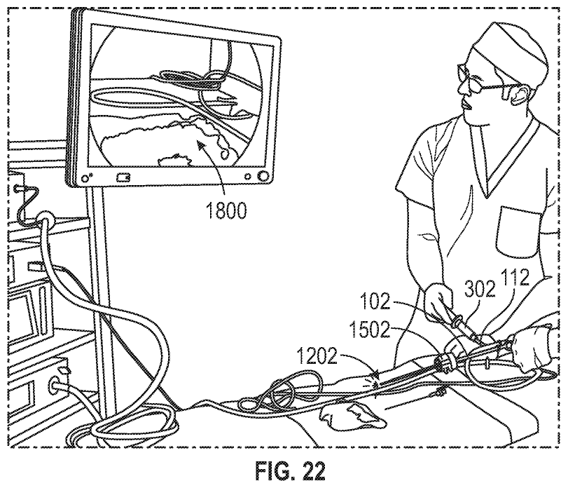

Referring now to FIGS. 19 and 20, when the field of view becomes obstructed, blurred, or otherwise blocked, cleaning fluid from the syringe 302 may be injected into the body 102. The cleaning fluid may flow through the fluid conduit 306 and out the distal end 204 of the body 102 and over the optics end 1202 of the endoscope 304. After the cleaning fluid is passed over the optics end 1202 the obstruction may be cleared away and the field of vision restored as shown in FIG. 22 with the need to remove the endoscope 304 from the body.

These and other embodiments for methods of creating a cleaning sheath may incorporate concepts, embodiments, and configurations as described above. The particular implementations shown and described are illustrative of the technology and its best mode and are not intended to otherwise limit the scope of the present technology in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

The description and figures are to be regarded in an illustrative manner, rather than a restrictive one and all such modifications are intended to be included within the scope of the present technology. Accordingly, the scope of the technology should be determined by the generic embodiments described and their legal equivalents rather than by merely the specific examples described above. For example, the components and/or elements recited in any apparatus embodiment may be assembled or otherwise operationally configured in a variety of permutations to produce substantially the same result as the present technology and are accordingly not limited to the specific configuration recited in the specific examples.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present technology, in addition to those not specifically recited, may be varied or otherwise particularly adapted to specific environments, manufacturing specifications, design parameters or other operating requirements without departing from the general principles of the same.

The present technology has been described above with reference to exemplary embodiments. However, changes and modifications may be made to the exemplary embodiments without departing from the scope of the present technology. These and other changes or modifications are intended to be included within the scope of the present technology, as expressed in the following claims.

1. A cleaning sheath for directing a fluid supply from a syringe to an optics portion of an endoscope, comprising:
a sheath having a lumen extending between an open proximal end and an open distal end, wherein:
the sheath is configured to receive the endoscope within the proximal end;
allow the optics portion to extend through the lumen to the distal end; and
a fluid conduit is formed completely around an outer surface of the endoscope and extending along the length of the sheath between the open proximal end and the open distal end;
a fluid hub, comprising:
opposing first and second open ends;
a hub lumen extending between the opposing first and second open ends, wherein:
the hub lumen forms an insertion path for the endoscope;
the proximal end of the sheath is positioned within and coupled to the second open end of the fluid hub; and
the hub lumen is in fluid communication with the fluid conduit; and
an inlet port open to the hub lumen and disposed between the first and second open ends; and
a fluid line comprising:
a first end coupled to the inlet port; and
a second end configured to be coupled to the fluid supply.

2. A cleaning sheath according to claim 1, wherein the fluid hub comprises a seal disposed within the first open end configured to create a seal between the outer surface of the endoscope and the hub lumen.

3. A cleaning sheath according to claim 2, wherein the seal comprises an o-ring.

4. A cleaning sheath according to claim 1, wherein the fluid line comprises a flexible tube extending between the first and second ends.

5. A cleaning sheath according to claim 1, wherein the second end of the fluid line comprises a luer lock.

6. A cleaning sheath according to claim 1, wherein the sheath is rigid between the proximal and distal ends.

7. A cleaning sheath according to claim 1, wherein the sheath comprises:
an inner wall;
an outer wall disposed around the inner wall; and
a gap disposed between the inner and outer walls to form the fluid conduit between the open proximal end and the open distal end.

8. A cleaning sheath according to claim 7, wherein:
the distal ends of the inner and outer walls are coupled together to form a seal; and
the distal end of the inner wall comprises a plurality of openings configured to direct the fluid supply from the fluid conduit inwardly towards the optics end of the endoscope.

9. A cleaning sheath according to claim 1, wherein the distal end of the sheath is angled between zero and ninety degrees.

10. A cleaning sheath for directing a fluid supply from a syringe to an optics end of an inserted endoscope, comprising:
a sheath having a lumen extending between an open proximal end and an open distal end and configured to form a fluid conduit completely around an outer surface of the inserted endoscope and extending along the length of the sheath between the open proximal end and the open distal end;
a fluid hub, comprising:
opposing first and second open ends;
a hub lumen extending between the opposing first and second open ends, wherein:
the hub lumen forms an insertion path for the endoscope;
the proximal end of the sheath is positioned within and coupled to the second open end of the fluid hub; and
the hub lumen is in fluid communication with the fluid conduit, wherein the first open end comprises a sealing element configured to create a seal between an outer surface of the inserted endoscope and the hub lumen; and
an inlet port open to the hub lumen and disposed between the first and second open ends; and
a fluid line comprising:
a first end coupled to the inlet port; and
a second end configured to be coupled to the fluid supply.

11. A cleaning sheath according to claim 10, wherein the sealing element comprises an o-ring.

12. A cleaning sheath according to claim 10, wherein the second end of the fluid line comprises a luer lock.

13. A cleaning sheath according to claim 10, wherein the sheath comprises:
an inner wall;
an outer wall disposed around the inner wall; and
a gap disposed between the inner and outer walls to form the fluid conduit between the open proximal end and the open distal end of the sheath.

14. A cleaning sheath according to claim 13, wherein:
the distal ends of the inner and outer walls are coupled together to form a seal to close off the fluid conduit; and
the distal end of the inner wall comprises a plurality of openings configured to direct the fluid supply from the fluid conduit inwardly towards the optics end of the endoscope.

15. A cleaning sheath according to claim 10, wherein the distal end of the sheath is angled between zero and ninety degrees.
